(19) United States
(12) Patent Application Publication
(10) Pub. No.: US 2024/0206376 A1
(43) Pub. Date: Jun. 27, 2024

(54) System and Method for Reducing Down-Leaching of Excess of Fertilization to a Below-Roots Zone

(71) Applicant: B.G. NEGEV TECHNOLOGIES AND APPLICATIONS LTD.,AT BEN-GURION UNIVERSITY, Beer Sheva (IL)

(72) Inventors: Ofer DAHAN, Midreshet Ben Gurion (IL); Elad YESHNO, Midreshet Ben Gurion (IL); Shlomi ARNON, Lehavim (IL)

(21) Appl. No.: 18/569,103

(22) PCT Filed: Jun. 12, 2022
(86) PCT No.: PCT/IL2022/050624

§ 371 Date: Dec. 11, 2023

Related U.S. Application Data
(60) Provisional application No. 63/210,020, filed on Jun. 13, 2021.

(51) Int. Cl. A01C 21/00; G01N 33/24

(52) U.S. Cl. CPC A01C 21/007; G01N 33/24; G01N 2033/245

(57) ABSTRACT

The invention relates to a system for reducing down-leaching of nitrate to a region below a crop's roots zone, comprising: (a) an analysis unit for repeatedly determining a concentration level of nitrate at least at a region below the crop's roots zone, and recording the nitrate concentration levels: (b) a controller configured to: (i) receive a recent record of the nitrate concentration level below the roots zone and at least one previous record of concentration level, and determine a rate of change between the recent and previous records: and (ii) based on the rate of nitrate concentration change, activating fertigation and irrigation in times and periods that minimize the down-leaching of nitrate to below the roots zone: wherein the system comprises at least one water-sample collecting sensor positioned below the crop's roots zone that transfers the sample to the analysis unit.

### FIELD OF THE INVENTION

The invention generally relates to systems and methods for optimizing agricultural crops' yields while reducing water contamination due to excess application of fertilizers. More specifically, the invention relates to a system and a method that optimize the fertilizers by the crop while minimizing the amount of nitrate percolating into the groundwater due to a possible excess of fertilization.

### BACKGROUND OF THE INVENTION

Contamination of rivers, lakes, freshwater, drinking water, groundwater, and soil pore water by nitrate is a global problem. The term “nitrate” is briefly referred to herein also as “N”. It is universally recognized that nitrate contamination of drinking water is a threat to human health. It is also reported that human health suffers from adverse effects, even cancer, due to continual exposure to nitrate above a certain level. A significant rate of water pollution results from excess fertilization by farmers due to a lack of real-time and accurate information regarding the nutrient availability in the soil and a lack of real-time monitoring of the excess fertilization given. Therefore, an excess of fertilization results in a waste of resources and pollution of the groundwater, particularly by nitrate. The terms “fertilization” and “fertigation” (which typically indicates fertilization via the irrigation system, where the fertilizer is mixed with water) are used herein interchangeably.

During the second half of the 20th century, clear trends of nitrate concentration increase in groundwater have been observed in aquifers all over the globe. The World Health Organization (WHO) has determined that nitrate levels in drinking water should not exceed 50 ppm. When exceeding this concentration level, nitrate is harmful to infants and can often lead to methemoglobinemia which also known as “blue-babies syndrome”, severe and illness even death. Unfortunately, nitrate contamination is the most dominant factor responsible for severely degrading groundwater and surface resources. On a global scale, eutrophication and hypoxia of streams, rivers, and lakes, are mainly attributed to subsurface return flow from nitrate contaminated groundwater, leaking from phreatic aquifers underlying agricultural fields.

Moreover, the impact of nitrate-contaminated groundwater is not limited it to terrestrial resources, water as significantly impacts also marine ecosystems. For example, eutrophication and hypoxia on a large scale have been found in the Gulf of Mexico and the Black Sea, and severe impacts on the Great Barrier Reef, Australia, have been observed. Overall, nitrate contamination had led to more groundwater disqualification and water well shutdowns than any other contaminant worldwide. While nitrate is considered the most common non-point source pollutant in groundwater, numerous studies have linked the increase of nitrate concentration in groundwater to the excess use of fertilizers in agriculture. As a result, a global regulatory takes place by environmental protection and water authorities to reduce the excessive application of agricultural fertilizers. For example, the European Union has established the Nitrates Directive, and the US Environmental Protection Agency (EPA) regards nitrate contamination in groundwater as an event requiring immediate action.

At present, fertilizer application in agriculture relies primarily on farmers' experience, expert recommendations, and sporadic soil testing. Unfortunately, none of these techniques provide information in line with the time scale of N-fertilizers mobilization (nitrate's solution movement rates through the soils sediments), consumption, and transformation dynamics in the soil.

Presently, the monitoring of chemical parameters in soils is performed in water samples that may be obtained, for example, by a suction cup mounted in the soil or by extracting soil samples. Water samples collected by this mechanism are typically transferred to a laboratory for further chemical analysis or analyzed on-site through an analytical kit. Moreover, nitrate concentration in the soil may fluctuate in time scales of hours to days due to different irrigation schemes, precipitation, fertilization, root uptake, and different plant growth phases. As such, conventional tools for measuring nitrate concentration do not meet the required time resolution for optimizing fertilization schemes while preventing groundwater pollution due to excessive fertilization. Moreover, current techniques typically require handling samples and laboratory analyses, which can be expected by a devoted research team, yet not by farmers.

Tuly et al. (2009), “In Situ Monitoring of Soil Solution Nitrate: Proof of Concept”, https://www.researchgate.net/publication/231523625, suggests a technique for continuous monitoring of nitrate concentrations in soil solution. Absorbance spectroscopy is applied to a sample within a stainless-steel porous cup installed in the soil. The porous cup is filled with deionized water and placed in a potassium nitrate solution reservoir. Once the solution inside the cup achieves chemical equilibrium with the surrounding solution resovobus by chemical diffusion, the absorption spectrum of the solution was measured through a UV dip probe, which was connected top a spectrophpto, ert and a UV light source. However, this technique has limited applicability for two main reasons: (a) the obtaining of chemical equilibrium between the porous cup and the surrounding medium, especially in unsaturated sediment with limited water storage, is relatively slow (hours to days), resulting in a time lag between the actual variation of the nitrate concentration in the soil to its actual measurement. Therefore, rapid concentration variations, as expected following intensive irrigation or fertilization events, may not be recorded; and (b) the presence of natural soil Dissolved Organic Carbon (DOC) limit the accuracy of UV absorption by employing spectroscopy analysis since both nitrate and soil DOC absorb UV light in overlapping wavelengths ranges.

Nitrate is highly chemically stable and mobile ion. As such, nitrate is leached down easily from the root zone by the rain and irrigation water and ultimately percolate through the unsaturated zone to the water table and contaminates the aquifers and related surface water sources. On a global average, root uptake by plants only utilizes about 50% of the implemented nitrogen fertilizer and therefore the rest of it transforms into nitrate, which ultimately reaches the groundwater table. Therefore, the development of an analytical system that includes accurate robust sensors for obtaining real-time information on nitrate concentration in the soil is essential for both optimizing fertilizers and prevent water resource contamination.

WO 2018/104939, Yeshno et al. suggest a nitrate concentration determination technique based on a continuous spectral analysis of soil porewater in an optical flow cell. The optical flow cell is fluidly connected to a porous interface which obtains a continuous flux of soil porewater. The absorption spectrum of the soil porewater is continuously recorded and analyzed to determine in real-time the nitrate concentration. The analysis involves a scan of the absorption spectrum of the soil porewater to identify a single optimal wavelength where DOC interference to nitrate measurement is minimal. However, despite the capability of the system to continuously measure nitrate concentration in-situ, this system failed short in showing how to ensure that the irrigation and fertilization are optimized such that no (or minimal) excessive nitrate arrives in the groundwater.

WO 2020/250226 (Arnon et al.) discloses a system that includes (a) a first illuminator configured to illuminate a sample within a cell by light in a first wavelength and a first photodetector for collecting the first-wavelength illumination, following the light passage through the sample; (b) a second illuminator configured to illuminate the sample within the cell by light in a second fluorescence-excitation wavelength, and a second photodetector for collecting illumination in a third fluorescence-emission wavelength from the sample. An analysis unit determines the nitrate+DOC impact on the absorption spectrum as measured by the first photodetector, and it further determines the DOC concentration based on the fluorescence emission as measured by the second photodetector. Based on the two determinations, the nitrate concentration is found. In similarity to WO 2018/104939, this system also failed short in showing how to ensure that the irrigation and fertilization are optimized such that no (or minimal) excessive nitrate arrives in the groundwater.

It is an object of the present invention to provide a system for continuously, in real-time, and in-situ monitoring nitrate excess in the soil and automatically optimizing the irrigation and fertilization to reduce such excess without harming the crops' development.

Other objects and advantages of the invention become apparent as the description proceeds.

### SUMMARY OF THE INVENTION

The invention relates to a system for reducing down-leaching of nitrate to a region below a crop's roots zone, comprising: (a) an analysis unit for repeatedly determining a concentration level of nitrate at least at a region below the crop's roots zone, and recording the nitrate concentration levels; (b) a controller configured to: (i) receive a recent record of the nitrate concentration level below the roots zone and at least one previous record of concentration level, and determine a rate of change between the recent and previous records; and (ii) based on the rate of nitrate concentration change, activating fertigation and irrigation in times and periods that minimize the down-leaching of nitrate to below the roots zone; wherein the system comprises at least one water-sample collecting sensor positioned below the crop's roots zone that transfers the sample to the analysis unit.

In an embodiment of the invention: for a grain crops, vegetable crops, and greenhouse crops, the below the roots water-sample collecting sensor is positioned at a depth of between 50 cm to 70 cm below the ground surface, and for trees, the below the roots sensor is positioned at a depth of between 80 cm to 100 cm below the ground surface level.

In an embodiment of the invention, the system further comprising at least one additional water sample-collecting sensor positioned at the crop's roots zone, the sensor further transfers water samples to the analysis unit for further determination of nitrate concentration at the roots zone, and wherein the controller further considerers the level of nitrate concentration at the roots zone in its times and periods management of fertigation and irrigation configured to minimize the flow of nitrate to below the roots zone.

In an embodiment of the invention, for grain crops, vegetable crops, and greenhouse crops, the water-sample collecting sensor within the roots zone is positioned at a depth 30 cm to 50 cm below the ground surface, and for trees, the sensor within the roots zone is positioned at a depth of between 40 cm to 60 cm below the ground surface.

In an embodiment of the invention, the system comprising at least one additional water sample-collecting sensor positioned above the crop's roots zone, the sensor further transfers water samples to the analysis unit for further determination of nitrate concentration above the roots zone, and wherein the controller further considerers the level of nitrate concentration above the roots zone in its times and periods management of fertigation and irrigation configured to minimize the down-leaching of nitrate to below the roots zone.

In an embodiment of the invention, for grain crops, vegetable crops, and greenhouse crops, the water-sample collecting sensor above the roots zone is positioned at a depth 10 cm to 30 cm below the ground surface, and for trees, the sensor above the roots zone is positioned at a depth of between 20 cm to 40 cm below the ground level.

In an embodiment of the invention, the system comprising a set of three water-sample collecting sensors, wherein for grain, vegetable crops, and greenhouse crops, the sensors are positioned at depths of 10 cm-30 cm, 30 cm to 50 cm, and 60 cm and 50 cm to 70 cm, and for fruit trees the sensors are positioned at depths of 20-40 cm, 40-60 cm, and 80 cm to 100 cm, respectively.

In an embodiment of the invention, the system further comprising one or more soil moisture sensors, each sensor provides soil water content data at each depth, respectively, for consideration in the management of the fertigation and j irrigation that minimizes the down-leaching of nitrate to below the roots zone.

In an embodiment of the invention, a plurality of sets of water sample collecting sensors are positioned along a crop field and wherein the system averages concentration results of a plurality of sensors, respectively, that are positioned at the same depths.

In an embodiment of the invention, each water-sample collecting sensor comprising a porous interface and wherein the water sample is transferred to an optical flow cell within the analysis unit utilizing a tube.

In an embodiment of the invention, the analysis unit operates in real-time.

In an embodiment of the invention, the system further comprising closed-loop fertigation and irrigation management.

In an embodiment of the invention, the analysis unit operates off-line.

In an embodiment of the invention, the system operates in two stages, as follows: during a training stage the system is operated in a closed loop to build a model defining a dependency of down-leaching of nitrate to below the roots zone on periods and amounts of irrigations and fertilizations, respectively; and during an operational stage, a control unit operates in an open-loop without sensors and analysis unit, u applying irrigations and fertilizations following a plan prepared based on said model, said plan is configured to minimize down-leaching of nitrate to a region below a crop's roots zone.

In an embodiment of the invention, the system further utilizes weather and rain data to optimize the management of fertigation and irrigation.

The invention also relates to a method for reducing down-leaching of nitrate to a region below a crop's roots zone, comprising: (a) positioning at least one water-sample collecting sensor below the crops' roots zone; (b) optionally positioning at least one additional water-sample collecting sensor at or above the crops roots zone; (c) receiving water samples from the water-sample collecting sensors, and repeatedly determining a concentration level of nitrate at least at a region below the crop's roots zone, and possibly also at the roots zone and above the roots zone, and recording the concentration levels; (d) based on the determination of nitrate concentration at least below the roots zone, and previous one or more recordings of nitrate concentration below the roots zone, determining a rate of concentration change below the roots zone; and (e) based on the rate of change, managing fertigation and irrigation in times and periods that minimize nitrate down-leaching to the region below the roots zone.

In an embodiment of the invention, the method includes further positioning one or more wetness sensors, at locations selected from below the roots zone, at the roots zone, and/or above the roots zone, and considering wetness data acquired by these sensors for the irrigation and fertigation management.

In an embodiment of the invention, the method further considers weather data for the management of irrigation and fertigation.

In an embodiment of the invention, the method, the management comprising: (a) determining a nitrate concentration below the roots zone, and optionally also at or above the roots zone, and recording the determinations; (b) comparing between a current nitrate concentration below the roots zone and a previous nitrate concentration determination below the roots zone and determining a rate of change in the nitrate concentration; (c) comparing the rate of change to a predefined threshold scale; (d) if the rate of change is found to be high, performing one or more of postponing, reducing, or skipping the next irrigation and/or fertigation; or (e) if the rate of change is found to be low or zero, continue the irrigation and fertigation according to a regular protocol.

In an embodiment of the invention, the method further considers nitrate concentration determinations at or above the roots zone to manage irrigation and fertigation.

In an embodiment of the invention, the method further comprising: during a training stage, applying the method in a closed loop and building a model defining a dependency of down-leaching of nitrate to below the roots zone on periods and amounts of irrigations and fertilizations, respectively; and during an operational stage, operating in an open-loop without positioning said one or more sensors, and without determining said nitrate concentrations and rate of change, while applying irrigations and fertilizations following a plan prepared based on said model, said plan is configured to minimize down-leaching of nitrate to a region below a crop's roots zone.

### BRIEF DESCRIPTION OF THE DRAWINGS

In the drawings:

FIG. 1 illustrates a structure of a real-time prior art system for determining a concentration of nitrate in soil;

FIG. 2a illustrates a general structure of the system of the invention;

FIG. 2b illustrates a general structure of the system of the invention, in a configuration that includes both water-sample collecting sensors and soil moisture sensors at various depths;

FIG. 3 shows a nitrate measurement at a 60 cm depth, below the roots zone, in an experiment made within a tomato's open crop field;

FIG. 4 illustrates a system that includes a plurality of sets of nitrate monitoring sensors spread over a large field;

FIG. 5 illustrates a method for minimizing excess fertilization, according to an embodiment of the invention;

FIG. 6 illustrates the structure of the system of the invention, as used during an experiment;

FIG. 7a shows a top, and FIG. 7b shows a cross-section view (A-A′) of the Optical Fiber Multiplexer (OFM) used in an experiment;

FIG. 8 illustrates in block diagram form a possible open-loop control of the invention's system;

FIG. 9 illustrates in block diagram form a closed-loop control of the invention's system;

FIGS. 10a-10c provide an overview of a controlled fertigation experiment. FIG. 10a provides the N-inputs to the soil, and continuous nitrate data at depths of 20, 40, and 60 cm; FIG. 10b provides the data relating to daily irrigation and soil moisture content at depths of 20, 40, and 60 cm; and FIG. 10c shows the wheat crop growth stage and its height throughout the growing cycle;

FIG. 11 shows Observed vs. predicted nitrate concentration for the controlled irrigation and fertilization experiment;

FIGS. 12a and 12b illustrate Phase I of the controlled irrigation and fertilization experiment: FIG. 12a shows N-inputs to the soil and continuous nitrate data from 20 cm, 40 cm, and 60 cm. FIG. 12b shows the daily irrigation and soil moisture content data at 20 cm, 40 cm, and 60 cm;

FIGS. 13a-13b show phase II of the controlled irrigation and fertilization experiment: FIG. 13a shows N-inputs to the soil and continuous nitrate data from 20 cm, 40 cm, and 60 cm. FIG. 13b shows daily irrigation and soil moisture content data at 20 cm, 40 cm, and 60 cm;

FIG. 14 shows water content in the soil at 20 cm and 40 cm and daily irrigation during phase II of the controlled irrigation and fertilization experiment;

FIGS. 15a and 15b illustrate the phase III of the controlled irrigation and fertilization experiment: FIG. 15a shows the N-inputs to the soil, and continuous nitrate data from 20 cm, 40 cm, and 60 cm; FIG. 15b shows daily irrigation and soil moisture content data at 20 cm, 40 cm, and 60 cm;

FIGS. 16a and 16b illustrate phase IV of the controlled irrigation and fertilization experiment: FIG. 16a shows N-inputs to the soil and continuous nitrate data from 20 cm, 40 cm, and 60 cm; FIG. 16b shows daily irrigation and soil moisture content data at 20 cm, 40 cm, and 60 cm;

FIG. 17 shows the nitrate levels, and soil water content at 20 cm during phase IV of the controlled irrigation and fertilization experiment; and

FIG. 18 generally illustrates in a flow-chart form a process for a real-time operation of the controller of the invention.

### DETAILED DESCRIPTION OF PREFERRED EMBODIMENTS

As noted, excessive fertilization is a major cause of water contamination globally. The prior art has suggested monitoring nitrate concentration in the soil to ensure that the crop receives the appropriate amount of fertilization. This nitrate monitoring involves introducing a suction cup (porous interface) underground, extracting a water sample from the soil, and determining the nitrate concentration in the water sample utilizing optical spectrum analysis. Various prior art systems, for example, the system disclosed in WO 2020/250226, can perform this determination automatically and in real-time.

Prior art systems for monitoring nitrate (either in real-time or otherwise) concentrate on the sufficient provision of nitrate to the plant; therefore, they inherently suggest positioning a single suction cup (hereinafter, also referred to as “sensor”) at the estimated plants' root zone. However, the inventors have realized that the prevention, even minimizing excess of fertilization, cannot be optimized by positioning a single sensor in the roots' zone, particularly as the nitrate in the soil is mobile, and its concentration dynamically changes within different underground soil depths. In addition, irrigations and fertilization contribute differently to the nitrate concentration in different depths in the soil. Therefore, a single suction cup at a single soil depth, even a network of suction cups, all within the roots' zone, is insufficient to prevent nitrate excess that can reach and contaminate the groundwater. The invention overcomes this deficiency by positioning at least one suction cup deep in the soil below the roots zone and managing the fertilization and irrigation based on readings from the deep sensor. Moreover, positioning additional sensors within or above the roots zone is preferable, as the additional sensors can contribute even more to the optimization task, namely to the reduction of excess fertilization.

Three different real-time systems for monitoring nitrate concentration in the soil are disclosed in WO 2018/104939 (Yeshno et al.), in WO 2020/250226 (Arnon et al.), and a co-pending PCT application claiming priority from US 63/210,018. By “real-time,” it is meant a system that can determine the rate of nitrate concentration in a sample even within a few seconds (following a non-real-time preparation period). Furthermore, it may take up to several hours (preparation period) from the time of the system's installation until a first nitrate concentration result becomes available. Following this preparation period, the determination of nitrate concentration becomes real-time. The invention's system is not limited to real-time nitrate determination, as it may also operate with non-real-time analyzers.

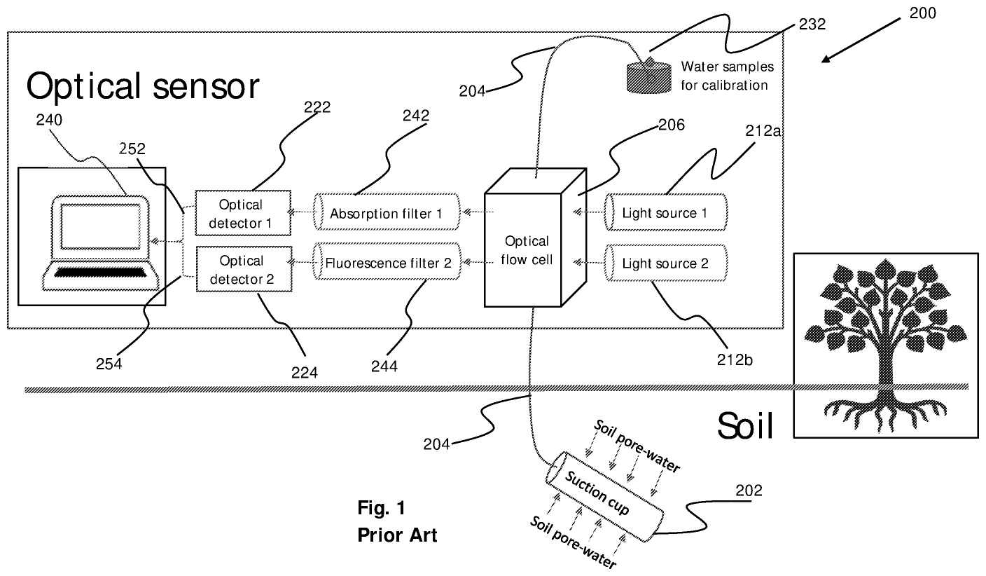

FIG. 1 schematically illustrates system 200, according to WO 2020/250226, and provides an example for a real-time system. A small volume porous interface (suction cup) 202, as disclosed, for example, in WO 2018/104939, is placed in the soil to obtain a continuous low flux stream of soil porewater solution. The soil porewater flows through tube 204 to an optical flow cell 206 via a small diameter tubing 204. The sample extraction from the soil is driven, for example, by applying low pressure (vacuum) on the porous interface. The sample can be later discharged or accumulated for further analysis or system calibration at sample accumulation chamber 232. The casing of flow cell 206 is at least partially transparent to allow passage of light beams therethrough. A first light source 212, preferably of LED type, illuminates the cell in a first UV wavelength, for example, in the proximity of 300 nm. The light beam of the first light source 212 passes through the optical flow cell 206, in which the soil porewater flows, while some of the light beam's energy is absorbed by the water constituents (the water contains nitrate and DOC). The remaining energy from the light beam of the first light source 212 is accumulated by photodetector 222, forming an absorbance signal 252. A processing unit 240 calculates an absorbance signal reflecting the difference between the illumination intensity by the first light source 212 and the light intensity accumulated by photodetector 222 (Beer-Lambert equation). A second light source 214, preferably of a LED type, illuminates the cell 206 in a second wavelength, for example, in an excitation wavelength near 350 nm. The light beam from the second light source 214 excites the DOC within the water sample, causing a DOC fluorescence emission at a secondary wavelength of, for example, 451 nm. The fluorescence emission results substantially only from the DOC, is proportional to the DOC concentration, and is independent of the sample's nitrate concentration. The fluorescence emission resulting from the second light beam is accumulated by the second photodetector 224, forming a fluorescence signal 254. The fluorescence signal 254 is conveyed to the processing unit 240, where a predetermined calibration equation is used to estimate the DOC concentration in the sample. As previously mentioned, the fluorescence emission intensity is in proportion to the DOC concentration in the solution. However, the fluorescence emission intensity is also proportional to the intensity of the excitation illumination by the second light source 214. Yet, the intensity of the excitation illumination is a parameter that the system operator controls. Optionally, filters 242 and 244 are located in front of optical detectors 222 and 224, respectively, to ensure the passage of only the wavelengths of interest towards the respective detectors. For example, a filter 244 allowing only light at 451 nm is located in front of the fluorescence detector 224, ensuring that the 350 nm light from the excitation beam is not received and does not saturate the detector 224 or mask the fluorescence reading. In addition, a filter for allowing only the passage of, for example, 300 nm is located in front of the absorbance detector 222 to ensure that the measurement is carried out at the zone where nitrate has a maximum absorbance peak. Other real-time systems, for example, those disclosed in WO 2018/104939 and co-pending PCT application claiming priority from US 63/210,018, are somewhat different in structure, but all utilize a single suction cup within the roots zone that cannot determine a flow of excess of nitrate to the groundwater.

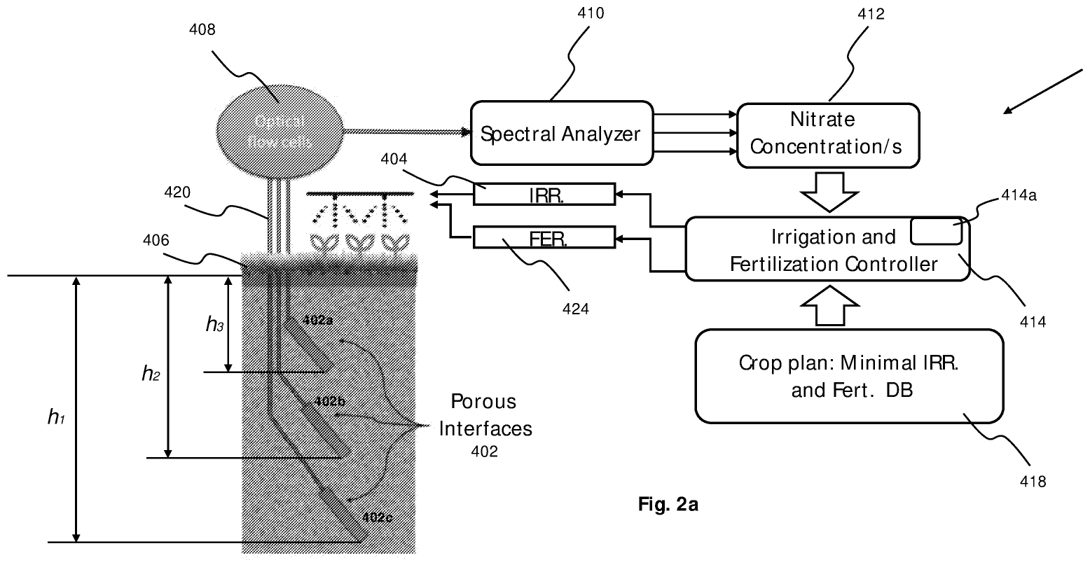

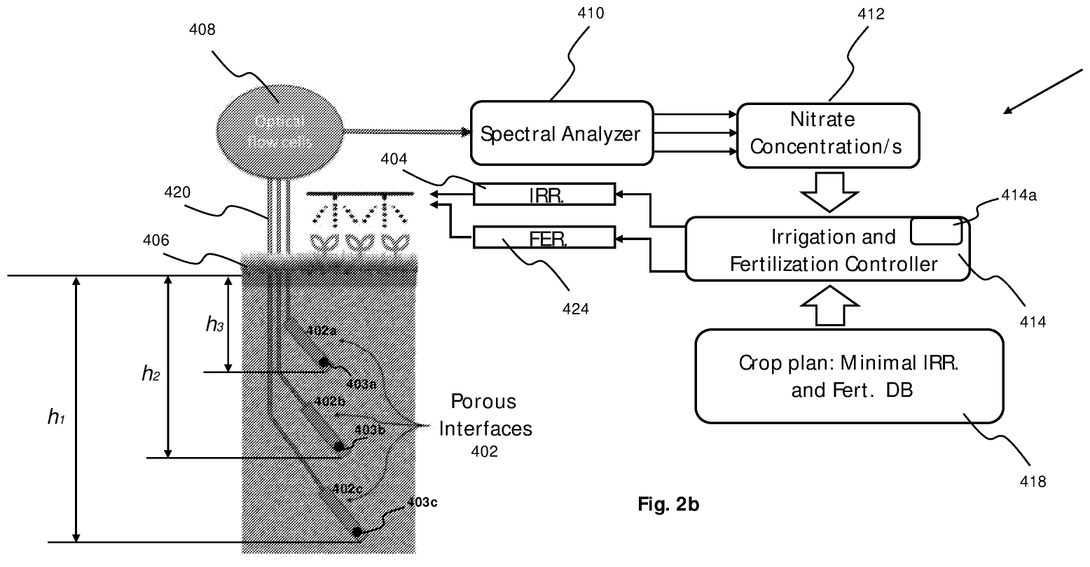

FIG. 2a illustrates a general structure of the system of the invention. The system includes at least one sensor 402c, positioned at depth h₁ below the roots zone of the plants. The system may include, in addition, one or more optional higher positioned sensors, for example, sensor 402b at depth h₂ within the roots zone and sensor 402a at depth h₃ above the roots zone, where h₁>h₂>h₃. Each sensor transfers its sample (typically a slow flow of sample) to a respective optical flow cell 408 (in this case, one flow cell is provided for each 402) via a respective tube 420, as described, for example, in WO 2018/104939. Spectral analyzer 410 determines (preferably in real-time) the nitrate concentrations 412, respectively, in each depth for which a sensor 402 is available. The measured nitrate concentration levels are conveyed to the irrigation and fertilization controller 414. The fertilization and irrigation controller also receives a plan for minimal irrigation and minimal fertilization that the specific crop needs and is expected to collect for its optimized development, as known from the literature and guides. Given the at least one nitrate concentration at the deepest soil position 402c, and its recent change (namely, calculated propagation of nitrate relative to the previous measurement), controller 414 activates the irrigation and fertilization actuators, respectively, to ensure providing the crops with the minimal requirements 418 while also minimizing any increase of nitrate concentration at depth h₁ due to excess of fertilization. It should be noted that plan 418 may indicate that the crop needs Xmm of water and Ycc of fertilization per week. However, the distribution of these amounts during the weekdays is open, and controller 414 manages the irrigation and fertilization actuators to (a) ensure that minimal requirements 418 are met in the long run; and (b) manages actuators 404 and 424 such that the excessive nitrate below the roots zone, as measured by sensor 402_{c}, is minimized. It should also be noted that the plant's capability to accumulate nitrate from below the roots is substantially zero, so all the nitrate measured by sensor 402c is substantially excessive, lost, and is expected to contaminate the groundwater. Moreover, as the crops' capability to collect fertilization is limited, overfertilization does not necessarily result in a higher yield, but it causes higher groundwater contamination.

Controller 414 may also utilize 3^{rd} party weather and rain data, including forecasting information in its management of the fertigation and irrigation that minimizes the flow of nitrate to below the roots zone.

As noted, at least one sensor (402_{c}) should be positioned at depth h₁ below the roots zone, as this is the minimum number of sensors by which controller 414 can operate to minimize excessive nitrate concentration below the roots zone. This task can be fulfilled by controller 414 estimating the nitrate concentration at the roots zone and above, given the knowledge about the fertilization already provided and the capability of the crop to collect fertilization within a given period. However, a sensing unit 402 with three sensors at different depths above the roots zone, within the roots zone, and below the roots zone is highly preferable. This multi-depths configuration best provides the controller 414 with the capability to determine nitrate distribution, gradients, and flow dynamics within the soil cross-section and with the capability to fine-tune the nitrate concentration levels within or above the roots zone before the nitrate arrives at the deepest sensor's 402, position, where it is too late to use it.

FIG. 2b shows a more preferred embodiment of the invention, where soil moisture sensors 403a, 403b, and 403c are added at each (or a part) of the nitrate sensors 402a, 402b, and 403c locations, respectively. Sensors 403a, 403b, and 403c provide system 400 more accurate details on the nitrate concentration and soil water content at each of the depths, namely, above the roots, at the roots, and below the roots. In such a manner, controller 414 has a broader view of the field situation and flow dynamics, enabling it to manage the irrigation and fertigation better.

Naturally, the depth of the roots zone is not constant during the entire cycle of the crop's cultivation. Therefore, using a 3-sensors configuration at three different depths is preferable. During the very early stage of planting, the middle sensor 402b may serve the purpose of the below the roots zone (that deepest sensor 402c serves). Moreover, different types of plants have roots at different depths. It has been found that for grain, vegetable crops, and greenhouse crops, the possible sensors depths are 20 cm (e.g., 10-30 cm), 40 cm (e.g., 30-50 cm), and 60 cm (e.g., 50-70 cm). For fruit trees, for example, the optimal depths are 30 cm (e.g., 20-40 cm), 50 cm (e.g., 40-60 cm), and 90 cm (e.g., 80-100 cm); however, the user may configure these depths based on the case circumstances.

The controller 414 is typically based on an internal program 414a that activates the irrigation and fertilization actuators based on the actual nitrate concentration/s 412 determinations, the actual crop plan 418, and prior knowledge on how the nitrate typically flows given specific irrigations and fertilizations. Program 414a may be developed using, for example, machine learning, existing databases, user learning, or intuition over time. The user may also manually operate the controller, given the actual concentrations and plan data 412 and 418.

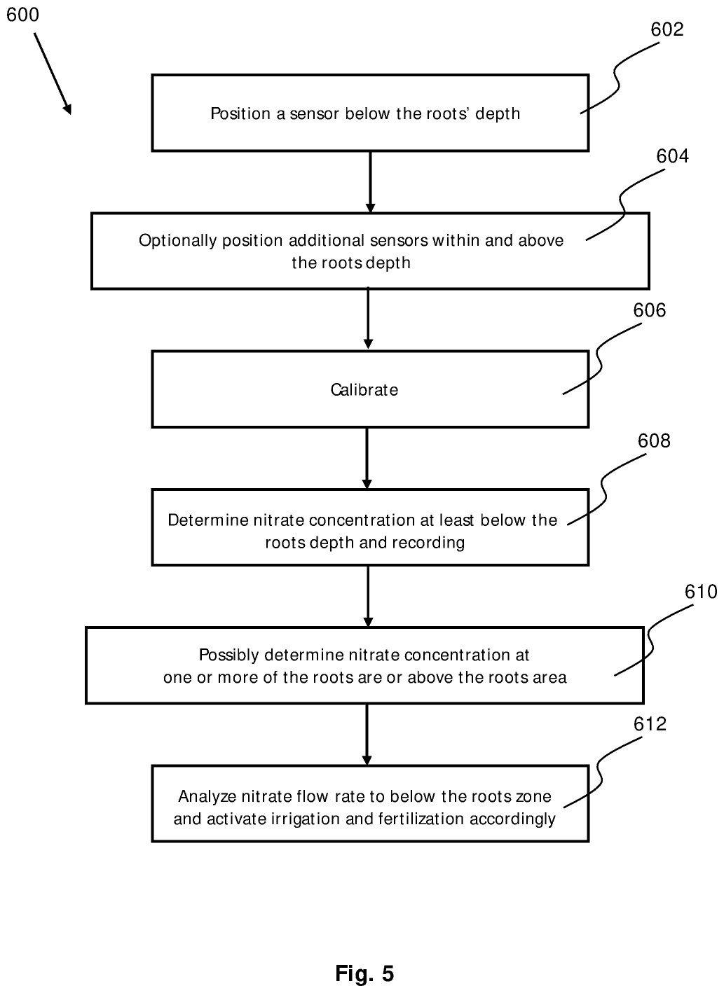

FIG. 5 illustrates a method 600 for minimizing the excess of fertilization, according to an embodiment of the invention. In step 602, a nitrate sensor is positioned at a depth below the roots zone, and the additional components of the system are installed, including controller 414, its program, and respective databases, as necessary. In step 604, additional sensors are optionally positioned within and above the roots zone. In step 606, the system is calibrated, for example, a calibration equation, as described, for example, in one of WO 2018/104939, WO 2020/250226, or a co-pending PCT application claiming priority from US 63/210,018 is determined. In step 608, the online operation of the system begins by determining the nitrate concentration below the roots' depth. Then, in step 610, the nitrate concentration in additional depths is obtained. Finally, in step 612, the nitrate flow rate to below the roots zone (by comparison with the previous recording), and based on said flow rate, the irrigation and fertigation are timely activated or terminated to minimize the flow rate to below the roots zone.

### Example 1

This example shows how optimization can be obtained using a single sensor below the roots zone.

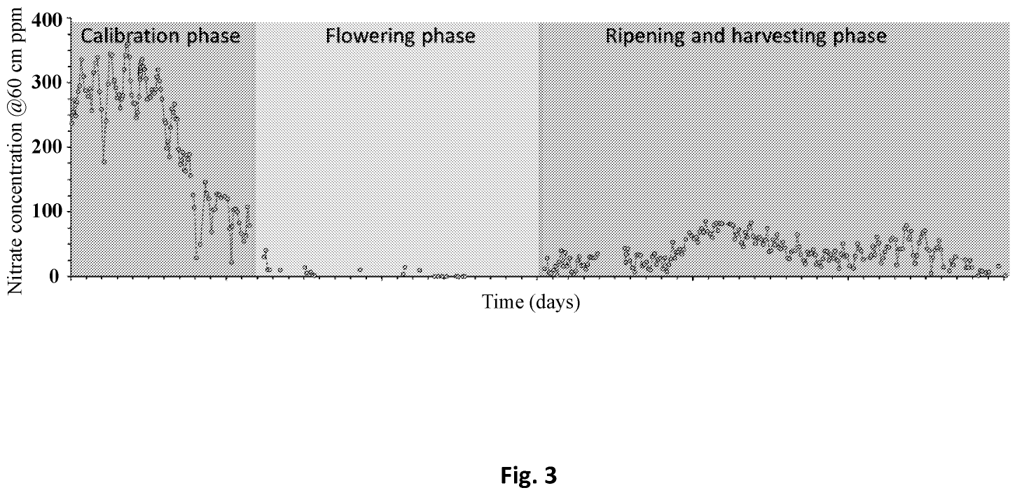

Generally, fertilizer application can be optimized by monitoring nitrate at a single point below the root zone. In the following scenario shown in FIG. 3, when measuring nitrate at 60 cm below the root zone of a tomato's open crop field, the effect of the irrigation and fertigation patterns was studied (calibration stage), achieving the knowledge required to perform the delicate fine-tuning for optimizing the application of those parameters. Later on, nitrate concentrations were optimized during the crop flowering growth stage. The optimal nitrate inputs were obtained using measurements at 60 cm depth (below the root zone of a tomatoes' open crop field), showing very low concentrations, between 14 to 40 ppm nitrate. Thus, it was concluded that the roots consumed all the applied nitrate substantially, and no leaching occurred. Subsequently, during the fruit ripening and harvesting phase, nitrate concentrations were brought up as recommended by fertilizers companies' guides, and as a result, nitrate levels below the root zone had increased and ranged in concentrations between 20 to 70 ppm nitrate. However, it should be noted that optimizing nitrate concentrations by a single sensor (402c) holds a more considerable risk of causing nutrient deficiencies than dual or triple depth nitrate monitoring. Measuring at more than one point provides the farmers and agronomists a complete picture of the soil profile and the temporal variations and gradients of nitrate concentrations in the soil profile.

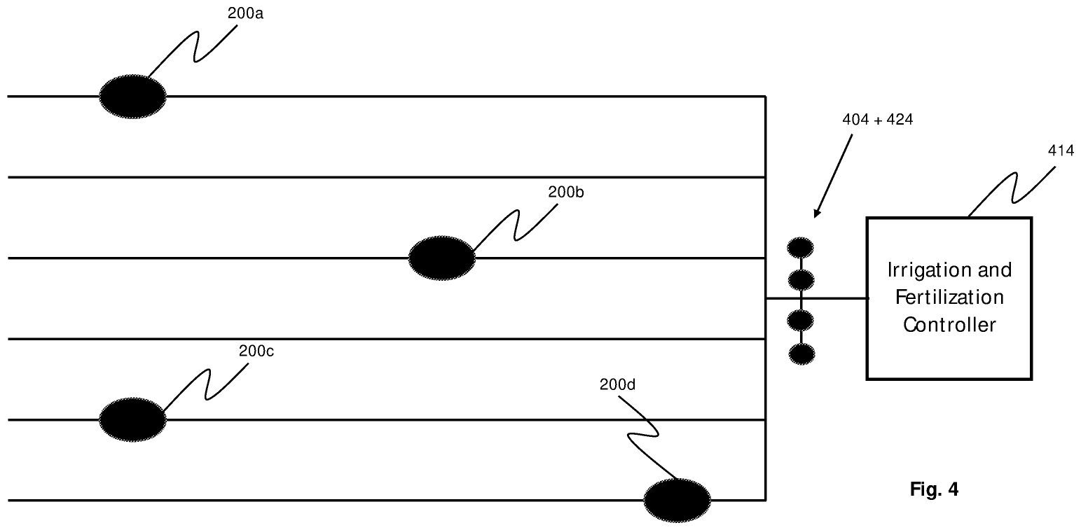

FIG. 4 illustrates how a field of, for example, 10 dunams can be controlled according to an embodiment of the invention. Controller 414 is positioned close to irrigation taps 404 and fertigation taps 424. One or more monitoring units 200a-200d are spread within the field, and each can determine the nitrate concentrations at one or more depths in a manner as detailed above. The monitoring units transmit their determinations to controller 414, which activates or deactivates the irrigation and fertigation taps for specific times. Controller 414 may typically average measurements of the same depths while controlling taps 404 and 424.

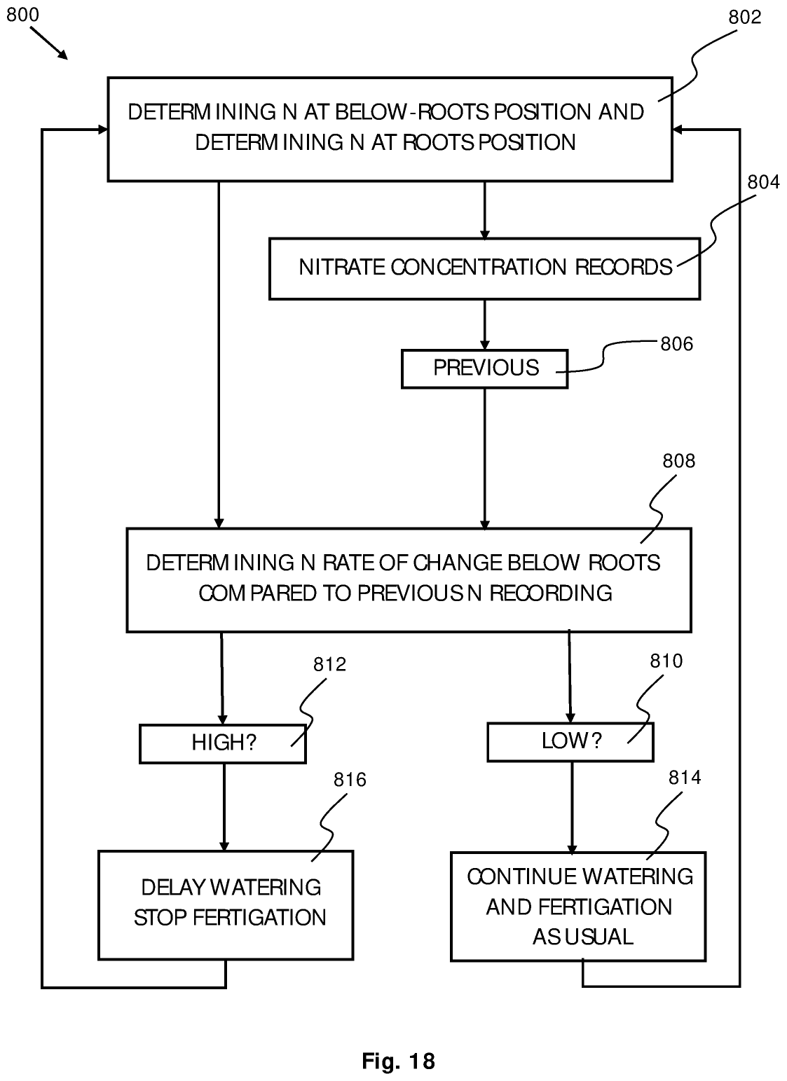

FIG. 18 generally illustrates a process 800 for a real-time operation of controller 414. Initially, the system 400 is installed, calibrated, and prepared for a real-time operation (steps omitted from FIG. 18). The installation includes at least two nitrate concentration sensors, a first sensor 402b (FIG. 2a) within the roots zone and a second sensor 402c below the roots zone. For simplicity, the following description of FIG. 18 assumes the installation of said two sensors (402b, 402c). In step 802, the nitrate concentrations within the roots zone (by sensor 402b) and below the roots zone (by sensor 402c) are determined and recorded in step 804. The recordings of step 804 keep respective records of the nitrate determinations of step 802. The determinations 802, respective recordings 804, and other decisions of process 800 may apply a predefined cycle of, for example, 1-day. The cycle may remain the same during the entire growing season or may be changed, for example, according to growth stages and crop developments. In step 808, and based on one or more previous recordings 804, the rate of nitrate change (current measurement 802 compared to previous recording 806) below the roots zone is determined. As previously mentioned, all the nitrate found below the roots zone is lost, reflects the excess of fertilization, and eventually arrives in the groundwater. If the rate of nitrate change in step 808 is determined to be high (compared to an expected predefined threshold scale), in step 816, the controller skips (or reduces) the next fertigation and also delays (or skips) watering to prevent (or reduce) additional flow of nitrate from the roots zone to the region below the roots zone. This form of control gives the crop more time to collect the fertigation solution. The procedure then returns to step 802 for another cycle. If, however, in step 810 it is determined that the rate of nitrate change in step 808 is low or zero (compared to an expected predefined threshold scale), in step 814, the controller typically continues with the watering and fertigation according to the existing watering and fertigation protocols, respectively. Remaining with the existing protocols allows s the plant to continue with its normal development and collection of fertigation while maintaining a low or zero nitrate flow to the below-roots zone. From step 814, the procedure returns to step 802 for the next cycle. As previously noted, the determinations of the nitrate concentrations at and/or above the roots zone are optional but preferable, as they provide the system most comprehensive view of the nitrate concentrations at various depths and better capability to minimize, even eliminate, the flow of nitrate to below the roots zone. The use of wetness sensors at one or more depths is also preferable for the same reasons.

As shown, procedure 800 reduces or even entirely eliminates the excess fertilization and flow of nitrate to the below-roots zone.

### FURTHER DISCUSSION AND EXAMPLES

The invention demonstrates a novel methodology for applying fertilizers that optimizes the nutrient availability in the soil, resulting in almost zero nitrate discharge to the environment. The method utilizes a closed-loop algorithm that continuously analyzes information on nitrate concentration and soil moisture in multiple depths of the soil cross-section. Off-line analysis, although less preferably, may also apply. The monitoring system obtains continuous online data regarding variations in nitrate concentrations at different depths (at least below the depth of the roots), utilizing sensors installed along the cross-section of the agricultural soil. Conventional moisture sensors may also be used in conjunction with the nitrate sensors. A dedicated algorithm analyzes actual relating to nitrate concentration distribution relative to variations in soil moisture to obtain the accurate state of nutrient presence and mobility in the soil profile. The algorithm outputs irrigation and fertigation recommendations or actual operations that ensure nutrient availability in the root zone while preventing excess nitrate transport towards regions below the root zone. The Soil Nitrate Monitoring System (SNS) and the irrigation and fertilization algorithm were tested in a large soil lysimeter used to grow wheat for an entire season from seedling to harvest (˜3 months). The monitoring sensors (402a-402c—FIG. 2a) were installed at three different depths to represent typical soil cross-sections of a typical model crop: (1) A shallow root zone (typically above the roots); (2) A mid-depth root zone; and (3) A region below the root zone. The trend in nitrate concentration in the upper two depths was used to monitor nutrient availability to the roots and assess the dynamic transport of nitrate in the subsurface. Data trends in nitrate concentration within the deeper region were used to assess nitrate leachate out of the root zone, namely to deeper sections of unsaturated zones and ultimately to groundwater. The various observations made during these experiments demonstrated that by careful decision-making, based on real-time data obtained from the soil regarding nutrient and water content, nitrate leaches out of the root zone can be dramatically reduced while still maintaining optimal and healthy crops.

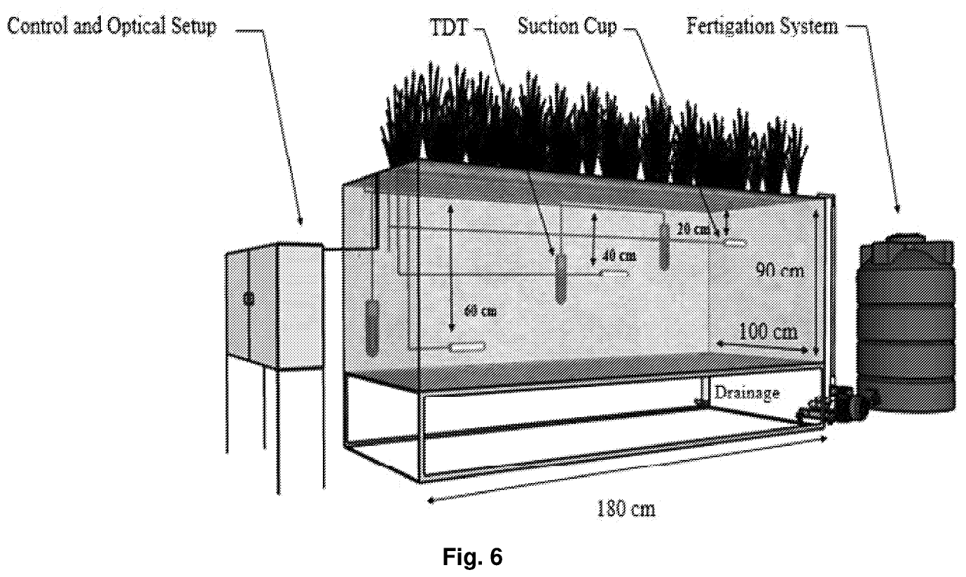

A controlled fertigation experiment was performed within a 1.0×1.8 m trapezoid-shaped lysimeter filled with fine sandy soil. The topsoil of the lysimeter was mixed with Dovrat Ltd. compost in recommended quantity by the Israeli Agriculture Bureau (1 in 3 per hectare). Three suction cups and three water content sensors (made by Acclima TDT) were placed in the soil at depths of 20, 40, and 60 cm (FIG. 6). Irrigation of the tank was conducted by a net of 49 drippers of fresh tap water, while a separate, 42 drippers net carried out fertilization. An average radial distance of about 20 cm was kept between the drippers to ensure uniform soil wetting during the experiment. The fertigation solution was created from “20-20-20 Haifa Poly Feed” fertilizer powder (5.7% of NO₃, 3.9% of NH₄, and 10.4% of NH₂ [Ureic Nitrogen]) mixture with tap water at a concentration range varying between 0.5-1.5 gr/L. The solution was held within a 100 L tank, ready for application via the fertigation dripper system. The experiment spanned wheat-growing through an entire growing season of 11 weeks, from seedling to harvest. The optical system's calibration, the wavelength choice, and the calibration equation were determined according to the protocol described in WO 2018/104939.

Continuous, in-situ measurement of the nitrate concentrations in various soil depths was determined by developing a monitoring apparatus in which the spectral absorption of the soil's pore water was measured within an optical flow-cell (206 in FIG. 1). The optical setup included a UV lamp and UV-VIS spectrometers set to measure light intensity between 190 nm and 850 nm, while a StellarNet SL3 deuterium light source was used as a continuous-wave UV light source. The spectrometer and UV lamp was connected to the flow-cell using optical fibers and collimating lenses (note that the optical fibers are optional, as practically the light source and the detector may be placed in front of the flow-cells directly). The porous interfaces (sensors 402a-402c) were connected to low-pressure (vacuum) sources via the optical flow-cells, respectively, to obtain continuous low flux streams of fresh soil porewater through the flow-cell. The sampling ports (232 in FIG. 1) were about 7 ml vessels located between the flow-cell and a vacuum bank. The samples at the sampling ports were collected and used to validate the results. More specifically, the respective nitrate concentrations were determined by a standard laboratory Ion chromatograph. The low pressure (vacuum) was used to form a slow stream (a few milliliters per hour) used to transfer the porewater sample, collected from the soil by the sensor (3 porous interfaces 402a-402c), towards the vacuum bank, via the optical flow-cell. The low pressure in the system was monitored and controlled by a set of pressure transducers connected to an Arduino-based control unit and a vacuum pump that was programmed to maintain a low pressure of 550-600 mbar within the sampling system. The porewater sample collection system was designed to function under small volumes of 4-6 mL by reducing the porous interfaces' inner volumes, using small-diameter tubing (inner diameter 1.6 mm) and low-volume flow-cells (about 1 ml). This low-volume design ensured the collection of fresh porewater solution to the optical flow-cells rapidly with minimal dilution along the solution transport path. As such, the measurement carried in the flow-cells represented the chemical characteristics of the soil's solution with a high level of accuracy.

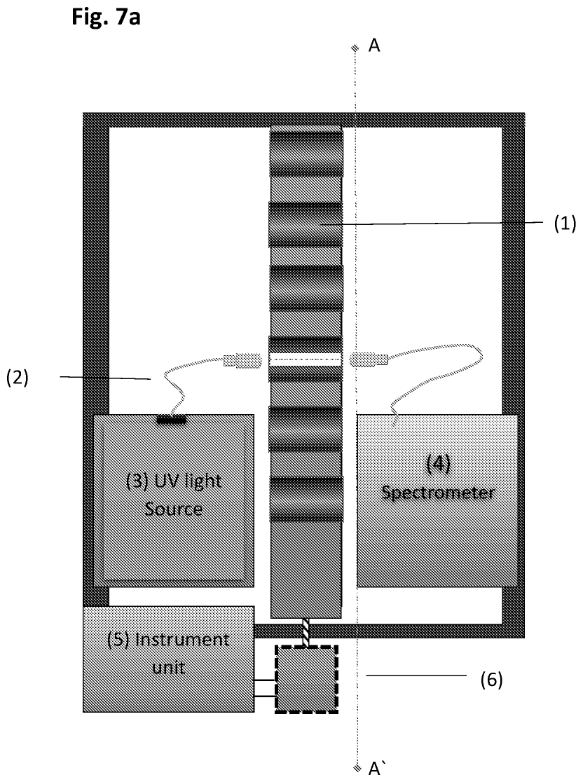

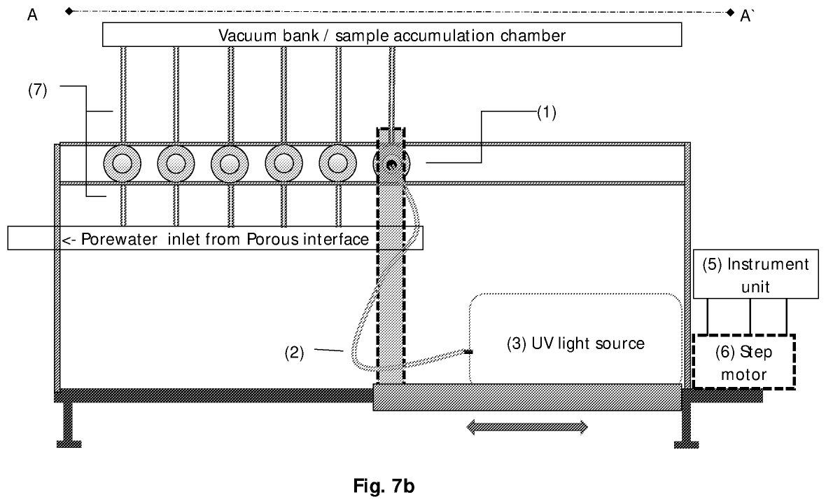

A dedicated optical fiber multiplexer was developed to enable a pair of single UV lamp and a spectrophotometer to measure the absorbance of an array of flow-cells. In its most general form, the Optical Fiber Multiplexer (OFM) is a mechanical control unit used to divert the UV beam between a plurality of optical flow-cells, allowing absorbance intensity measurement from multiple points/locations, as shown in FIGS. 7a and 7b. FIG. 7a shows a top, and FIG. 7b shows a cross-section view (A-A′) of the OFM unit. The OFM main system included: A set of optical flow cells (1); optical fibers and collimating lenses (2); UV light source (3); spectrometer (4); instrument unit (5); step motor (6); and inlet and outlet flow-cell piping (7). The OFM used a highly accurate step motor and a leading screw to move the optical fiber, UV lamp, and spectrometer to place it in front of a selected (stationary) optical flow cell from the set of flow cells 1.

The Lambert-Beer equation defines the absorbance intensity:

$$Absorbance = - {log}_{10} \frac{I}{{I}_{0}} \qquad ( 1 )$$

I indicate the light intensity after passing through the examined solution, and I₀ is the light intensity after passing through a reference sample (blank). However, since the UV lamp degrades with time, temperature variations affect the transmissivity of the optical fiber, causing additional signal intensity fluctuations; a decay in the measured signal might be interpreted as a false reading of the nitrate absorption by the solution. Therefore, a drift correction must be made to compensate for these signal intensity fluctuations. Measuring the pure intensity of the lamp could have been used to deduct intensity fluctuations from the measured light absorbance in the examined solution. However, in this experiment, the absorbance measurement drift was corrected utilizing the ability of the OFM to enable light intensity measurement at an array of flow-cells. The OFM was designed in a way that the default position of the ray enables the measurement of the UV beam when it is not passing through a flow cell or a solution (bypass route). As such, the intensity of the UV lamp is measured before each absorbance measurement session. The drift is then corrected by equations (2):

$$Absorbance = - {log}_{10} \frac{I}{{I}_{lamp}} \qquad ( 2 )$$

Where I indicate the light intensity after passing through the examined solution, and Iₗₐₘₚ is the current of the UV lamp as measured in the OFM bypass position.

The system of the invention may use open-loop or closed-loop control.

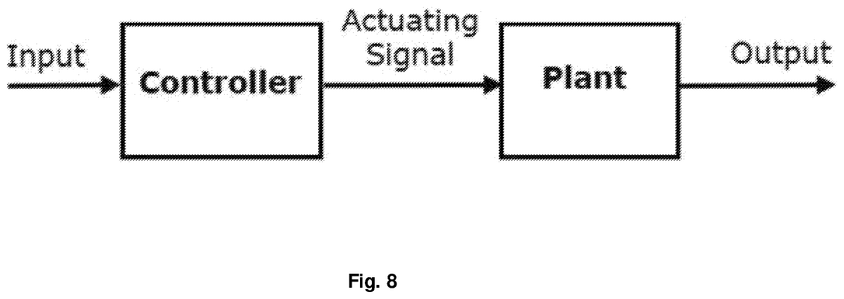

In a possible open-loop control, the output is not fed back to the input. Thus, the control action is independent of the desired output. FIG. 8 illustrates a possible open-loop control of the invention's system in block diagram form. The input is the nitrate, and the fertilization (or fertigation) is the controller and the actuator. The plant (controlled object) affects the relationship between an input signal and the feeding system's output signal. The output is the nitrate concentration at the measuring point at the root's zone.

The implementation of an open-loop system may include two phases:
(a) A training phase: sensors are positioned at different depths within the soil during the training phase. Then, a known volume of nitrate is injected into the soil (could be several concentrations and several volumes). Monitoring the change of concentration as a function of time at all locations provides the data required to build a model of nitrate propagation in the soils.
(b) The operational phase: during this phase, the outcome of combining the plant's consumption model of nitrate, as known from the literature, and the model developed in the training phase, are both used by the control system to calculate the nitrate required by the plant. Based on this calculation, the control system switches on and off the fertilization and the irrigation system to minimize the excess nitrate consumption. This control considers, among others, the plant's growing stages.

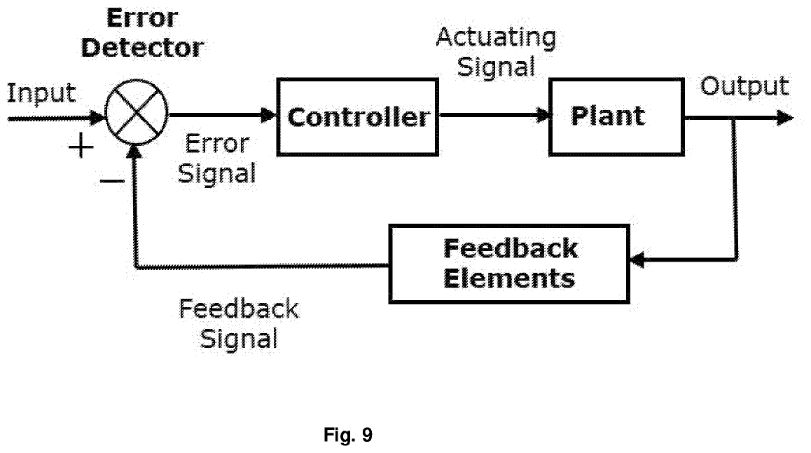

In a possible closed-loop control, the input is provided to a controller, producing an actuating or controlling signal. Then, this signal is supplied as input to a plant or to the process supposed to be controlled. So, the plant (control theory) produces an output, is the which controlled nitrate concentration. In this specific case, the concentration of nitrate below (and possibly also within) the root's zone is controlled. The respective measured concentration of nitrate below, within, or above the root zone, respectively, is fed back to the input. As such, the controlled action depends on the desired concentration level, as shown in FIG. 9.

The error detector produces an error signal, reflecting the difference between the input and the feedback signal. The feedback signal provided from the feedback-elements block is sampled from the output. Rather than direct input, the error signal is applied as an input to the controller. In the present case, the input is the nitrate, and the fertilization and irrigation sets are the controller and the actuator. The plant (controlled object) indicates the relationship between an input signal and the system's output signal within the soil. The feedback element is the signal generated by the buried nitrate sensor in the soil.

One way to implement the control system is by a PID (Proportional-Integral-Derivative) controller. A PID controller continuously calculates an error value e(t) as the difference between the desired setpoint and a measured process variable (in the present case, the nitrate concentration at, above, or below the root zone, respectively and applies a correction based on proportional, integral, and derivative terms. As implied by its name, PID (Proportional-Integral-Derivative) refers to the three terms operating on the error signal to produce the control signal. For example, let's assume that u(t) is the control signal sent to the system, y(t) is the measured output, and r(t) is the desired output, where e(t)=r(t)−y (t) is the tracking error. The PID controller has the general form of equation (3):

$$u ( t ) = {K}_{p} e ( t ) + {K}_{I} \int e ( τ ) d τ + {K}_{D} \frac{d e ( t )}{d t} \qquad ( 3 )$$

The desired closed-loop dynamics are obtained by adjusting the three parameters KP, KI, and KD, often iteratively by “tuning” and without specific knowledge of a plant (control theory) model. Stability can often be ensured using only the proportional term. The integral term permits the rejection of a step disturbance (often a striking specification in process control). The derivative term is used to provide damping or shaping of the response. PID controllers are the most well-established class of control systems.

In the present case, the three parameters KP, KI, and KD are adjusted according to the nitrate requirements for the growing stage of the plant, based on a literature model.

In another control system implementation, the inventors used a deep learning network as the control system. The sensors are positioned at different depths within the soil during the training phase. Then, a known volume (or several concentrations and several volumes) of nitrate is injected into the soil. Monitoring the concentration change as a function of time at all locations provides the data required to train the network.

In another implementation of closed-loop control, the parameter and the mathematical function of the controller are derived based on the literature and a training phase: sensors are positioned at different depths within the soil during the training phase. Then, a known volume of nitrate is injected into the soil (could be several concentrations and several volumes). Monitoring the rate of change of the concentration as a function of time at all locations provides the data required to build a model of nitrate propagation in the soils.

DOC and total nitrogen (TN) in the porewater samples from the soil were determined by an Analytic Jena multi-N/C 2100s TOC/TN analyzer. The nitrate concentration was determined by a Dionex ICS 5000 Ion chromatograph. The chemical and optical data of the porewater solution was analyzed by a MATLAB 2019b curve fitting tool to obtain the polynomial equation for nitrate estimation, correlation coefficient (R²), and RMSE values. The interference from the DOC was eliminated by an optimal wavelength calibration procedure, as described in WO/2018/104939.

Then, a procedure to determine the optimal wavelength calibration was performed. This procedure was designed to cope with the interference of the DOC to the nitrate analyses and is performed by UV absorption spectroscopy in porewater samples taken from the cultivated soil. At the core of this procedure is an algorithm that scans the absorption spectrum of a series of porewater solutions taken from a specific soil with variable concentrations of nitrate and natural DOC to locate an optimal wavelength, where the DOC interference to nitrate measurement is minimal, and the correlation to nitrate concentrations is maximal. This procedure was found effective for DOC concentrations up to 15 ppm. Although this analytical procedure is a site-specific feature, the calibration equations were stable for long periods and were successfully tested on porewater samples collected at four agricultural sites within two years.

The experiment primarily focused on measuring the down-leaching of nitrate from the root zone to the deeper unsaturated zone under various fertilization approaches. The last growing phase was used to develop an optimization algorithm capable of reaching zero down-leaching of nitrate while ensuring the necessary nutrient and water for the crop to achieve optimal yield.

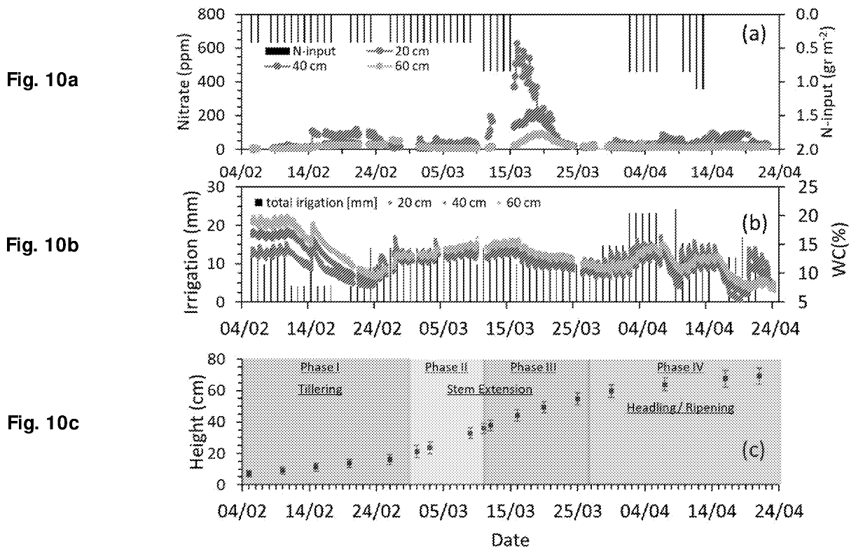

Throughout the experiment, attempts to control nutrient transport and its retention within the root zone of the cultivated soil were performed by intervening with both the irrigation and fertilization cycles. The irrigation and fertilization regimes' decision-making was based on real-time information on nitrate concentration and water content in the soil cross-section, as shown in FIGS. 10a and 10b. FIGS. 10a-10c provide an overview of the controlled fertigation experiment. FIG. 10a provides the N-inputs to the soil and continuous nitrate data at depths of 20, 40, and 60 cm; FIG. 10b provides the data relating to daily irrigation and soil moisture content at depths of 20, 40, and 60 cm; and FIG. 10c shows the wheat crop growth stage and its height throughout the growing cycle.

As such, the tested scenarios for irrigation and fertilization do not necessarily represent standard or recommended practices for wheat. Nevertheless, they represent different fertilization regimes that are often used in agriculture. The tested fertilization scenarios presented herein can be divided into four phases of irrigation and fertilization approaches shown in FIG. 8c. The first phase of the experiment was carried out while the crop was at its tillering stage and was characterized by uniform daily application of fertilizers with variabilities made in the fertilization regime. During this phase, where the plants are young, and roots are short and shallow, the irrigation and fertilization were reduced when the nitrate increased at a depth of 20 cm. In this stage, it was evident that the nitrate below 20 cm depth would not be utilized efficiently by the plant. As a result, any increase in the nitrate concentration in deep sections meant nitrate leaching with the percolating water.

The second phase took place during the crops stem extension stage. During this phase, an attempt to increase fertilizer retention in the topsoil and promote nitrate root uptake was performed by separating the irrigation and fertilization cycles. Thus, the nitrate mobilization was reduced by reducing the irrigation fluxes, and nitrate leaches. The third phase of the experiment also occurred during the crop stem extension stage to investigate the effect of intensive pulses' of fertilizers' application, as commonly practiced during agricultural activity. The fourth and last phase of the experiment was carried out during the crop headling and ripening stage. In this phase, daily adjustments to the fertilizers and water applications were managed based on the actual trends in nitrate concentration and variation in the soil water contents due to nitrate propagation. These fine adjustments to the irrigation and fertilizers input were made, given continuous information on nitrate concentration and water availability in the soil profile.

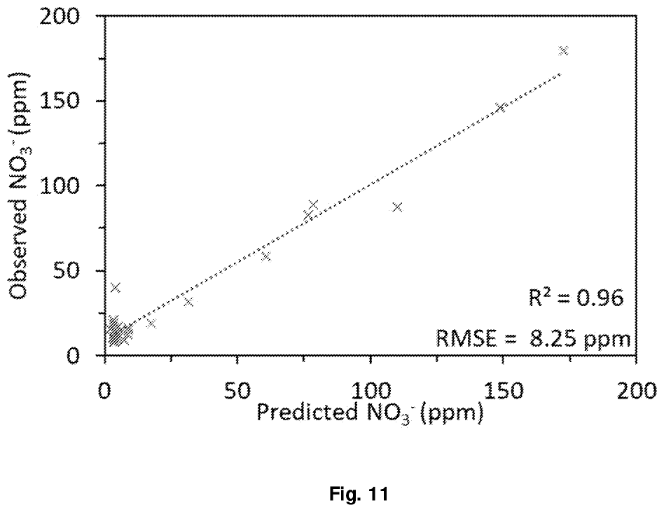

Validation of the measured results obtained by the SNS was carried out during the experiment through porewater samples that were collected and analyzed utilizing standard laboratory procedures for nitrate and total organic carbon. A comparison between the automated nitrate measurements (as obtained by the nitrate monitoring system) and the laboratory analyses were used to validate the integrity of the monitoring system. The validation test showed adequate results with R²=0.96 and RMSE=8.25 ppm, as shown in FIG. 11. FIG. 11 shows Observed vs. predicted nitrate concentration for the controlled irrigation and fertilization experiment.

### Phase I—Uniform Daily Fertilization, with Variabilities in the Irrigation Regime

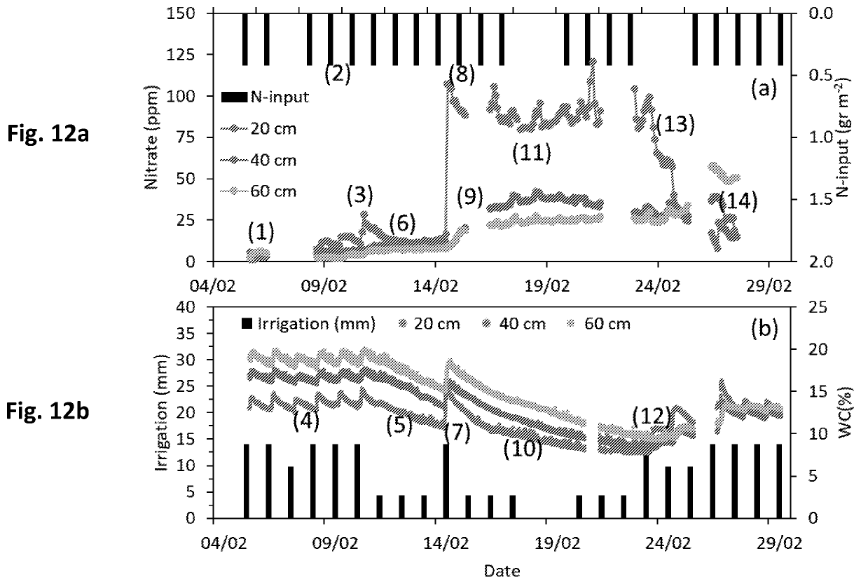

FIGS. 12a and 12b illustrate Phase I of the controlled irrigation and fertilization experiment: FIG. 12a shows N-inputs to the soil and continuous nitrate data from 20 cm, 40 cm, and 60 cm. FIG. 12b shows the daily irrigation and soil moisture content data at 20 cm, 40 cm, and 60 cm. At the initial phase of the experiment, an attempt was made to control nutrient transport in the soil cross-section by altering the daily irrigation cycles while keeping the daily fertilization cycle reasonably constant. The idea behind this scheme was to reduce the down leaching of nitrate, which occurs due to the irrigation events in the early stages when the plant's root system is not well developed. Thus, the concentrated nitrate solution is retained in the shallow soil, where the plant roots can utilize the nitrate. Since the wheat plants were at the tillering stage (FIG. 10c), the inventors expected that a low irrigation will align with the crop's demand for water. At the early stage of this phase of the experiment, nitrate levels in the soil did not exceed the typical concentrations found in the irrigation water (point 1 FIG. 12).

Yet, following the steady daily inputs of N-fertilizers (point 2 in FIG. 12), a gradual increase in nitrate concentration was visible at 20 cm, with a mild increase in nitrate concentration also at 40 cm and 60 cm (point 3 in FIG. 12). During this stage, the soil was irrigated on a daily basis. As a result, daily fluctuations in the water content at all three measured depths were observed, indicating continuous water percolation through the entire soil profile (point 4 in FIG. 12). In an attempt to reduce the potential of nitrate leaching, the irrigation was reduced to 4.2 mm per day for three days (point 5 in FIG. 12); no changes were made, however, to the daily fertilization. Following this step, a reduction in nitrate concentration was observed at 20 cm depth, while nitrate levels at 40 cm and 60 cm stabilized at about 20 ppm (FIG. 12). When the soil water content had dropped below 10%, 15 mm irrigation was applied to prevent plant stress from water deficiencies (point 7 in FIG. 12). As a result, a sharp increase in the nitrate concentration at 20 cm depth was noted (point 8 in FIG. 12). This sharp increase possibly resulted from the propagation of nitrate that accumulated in the shallow soil during the previous period of low irrigation regime. Additionally, an increase in nitrate levels was also observed at 40 cm and 60 cm depths, implying a possible nitrate down leaching (point 9 in FIG. 12). In the attempt to prevent additional nitrate leaches, the irrigation was brought to a secondary halt (point 10 in FIG. 12). During this period, nitrate levels at 20 cm had remained relatively high (about 100 ppm) and stable. Yet, at 40 cm, the nitrate showed a gradual increase until about 40 ppm, followed by a moderate reduction in nitrate levels to about 25 ppm nitrate (point 11 in FIG. 12). The nitrate concentration at 60 cm depth also showed a gradual increase during the same period. However, at this depth, nitrate levels did not drop; on the contrary, nitrate concentration was rising yet at a very moderate rate. It should be noted that during this period, the fertilization system did not work for a couple of days as a result of a mechanical failure—however, no significant changes in nitrate concentration resulting from the failure could be observed. From this point, when water content had dropped below 9%, it was decided to initiate daily irrigation cycles of 15 mm to prevent further plant stress in the continuance of this experiment (point 12 in FIG. 12). The following irrigation events caused significant nitrate leachate, expressed as a rapid decrease in nitrate concentration at 20 cm (point 13 in FIG. 12). Subsequently, the irrigation events led to the arrival of nitrate at 60 cm and a further decrease of nitrate levels at 20 cm (point 14 in FIG. 12). This observation implies that the nitrogen added to the soil during the tillering stage, when the roots were shallow and not well developed, moved down below the shallow soils where the plant could no longer consume it. Nevertheless, at the end of this phase (point 14 in FIG. 12), a reduction in the nitrate concentration was also observed at a depth of 40 cm and 60 cm, probably due to down leaching.

### Phase II—Separation Between Irrigation and Fertilization Cycles

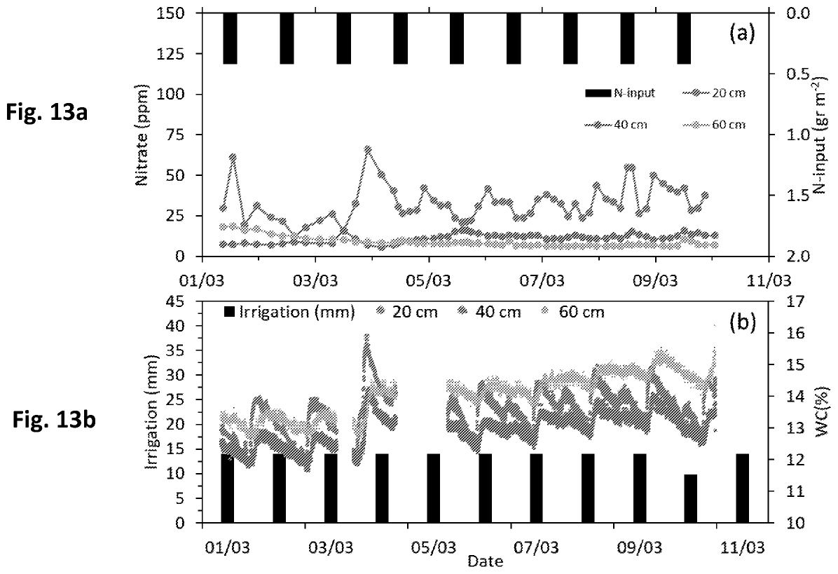

Following the substantial amount of nitrate leaching out of the root zone, which occurred during the tillering phase, the inventors concluded that it is impractical to control nitrate transport solely by controlling the daily irrigation. Therefore, the inventors hypothesized that increasing nitrate retention in the upper soil would enable more time for root uptake and reduce down leaching of nitrate below the root zone. Consequently, they initiated a separation between fertilization and irrigation to increase the time needed for nitrate to be consumed before the daily irrigation can drive leaches down. In the course of this phase, during morning hours, the soil was fertigated with 4.26 mm of 100 ppm N-fertilizer solution, enriching the topsoil with 0.42 gr m⁻² of nitrogen (FIG. 13a). FIGS. 13a-13b show phase II of the controlled irrigation and fertilization experiment: FIG. 13a shows N-inputs to the soil and continuous nitrate data from 20 cm, 40 cm, and 60 cm. FIG. 13b shows daily irrigation and soil moisture content data at 20 cm, 40 cm, and 60 cm. After a 12-hour delay, 9.8 mm of irrigation was subsequently applied to the soil (FIG. 13b).

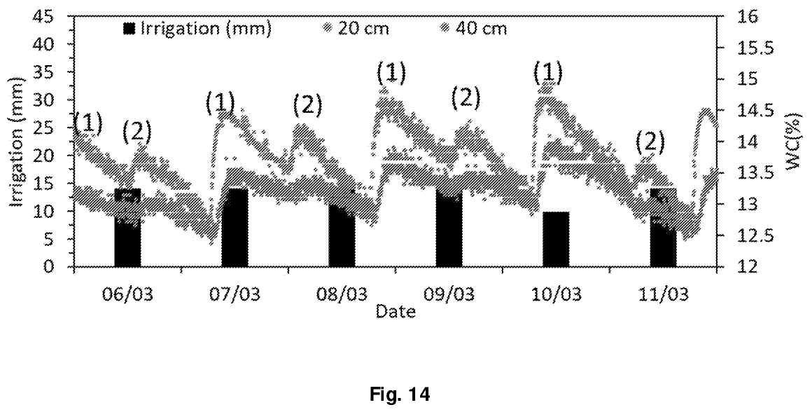

The time lag between the irrigation and fertilization cycles was visible through the daily variations in the measured water content, with a first peak associated with the fertigation event (points (1) in FIG. 14) and a secondary peak associated with the water irrigation (points (2) FIG. 14). FIG. 14 shows the water content in the soil at 20 cm and 40 cm and daily irrigation during phase II of the controlled irrigation and fertilization experiment. The low nitrate levels that remained at 40 and 60 cm at this phase of the experiment indicated that the applied nitrate quantity was uptake into the plant roots, and as such, no leaches were driven by the irrigation cycles. Additionally, it was determined that water irrigation does arrive in the deeper sections of the soil, as daily fluctuations in the water content levels appear very clearly at 40 cm and 60 cm as well (FIG. 13b). At this phase of the experiment, the crop was found at the steam extension stage (FIG. 10c) and had a higher demand for nutrients. As such, daily oscillations in nitrate levels at 20 cm could be seen, both due to the plant roots uptake and the daily fertilizer application (FIG. 13a). The direct outcome of this stage is that the task of reaching a substantially zero down-leaching of nitrate from the soil is possible and not out of reach.

As known in the art, the recommended supply of N-fertilizer to a wheat crop stands between 20 to 50 gr/m², when gradually applied through the growing season. Such a gradual application of nutrients can be achieved by adjustments made to the fertigation solution. However, some crops cannot be fertigated, mainly for practical reasons, such as rain-fed open crop fields for maze and wheat, and organic agriculture mainly based on manure for nutrient supply. These crops receive their nutrient supply mainly during the soil preparation, as in the case of organic farming, or through sporadic fertilizing events throughout the growing season.

### Phase III—Intensive Pulse Application of Fertilizers

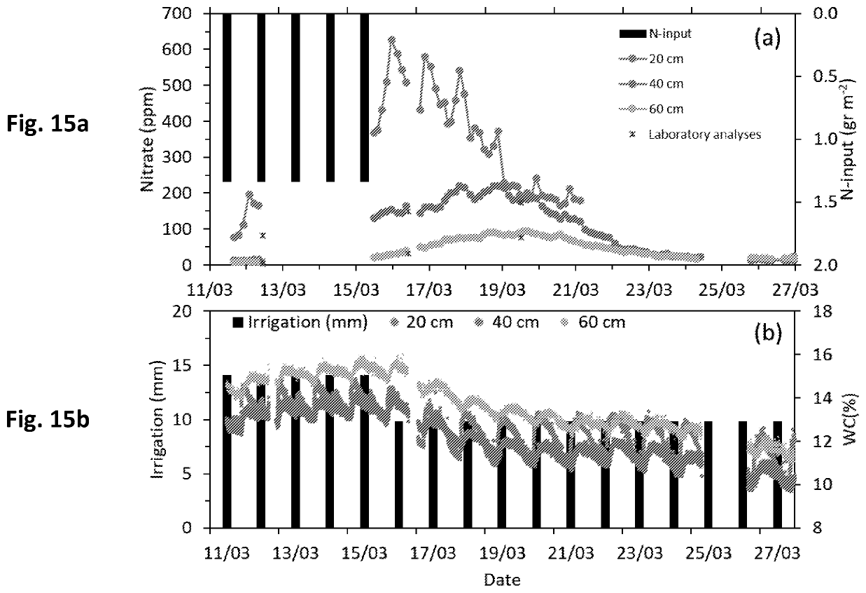

To investigate the effect of intensive fertilization events on the nitrate propagation (transport) below the root zone, the inventors programmed the fertigation system to supply rapid and intensive doses of fertilizer during a short period. This fertigation event was done during the stem extension phase, where nitrogen demand by the plant is relatively high, and the root system is already developed. Since during the above phases I and II of the experiment, a total amount of 8.8 gr/m² N-fertilizer was already supplied to the crop, it was decided not to exceed a total of 7 gr/m² N-fertilizers input throughout this phase. Additionally, to reduce nitrate contamination during the following stages of the experiment, this test was scheduled for performance at a point where the crop was at its stem extension stage and thus where a greater demand for nitrate exists. During this phase, the irrigation system was programmed to a daily cycle between 7-14 mm (FIG. 15b), and a daily dose of 1.4 gr/m² nitrogen was applied to the soil for 5 days (FIG. 15a). FIGS. 15a and 15b illustrate the phase III of the controlled irrigation and fertilization experiment: FIG. 15a shows the N-inputs to the soil, and continuous nitrate data from 20 cm, 40 cm, and 60 cm; FIG. 15b shows daily irrigation and soil moisture content data at 20 cm, 40 cm, and 60 cm. The intensive fertilization event had caused a rapid rise in nitrate concentration with peak nitrate values of 600, 200, and 100 ppm at 20 cm, 40 cm, and 60 cm, respectively (FIG. 15a). Despite the daily irrigation and the rapid peak values on the fifth day, the complete reduction of the nitrate concentration due to plant uptake or down leaching from the soil cross-section took about 8 days.

### Phase IV—Soil Nitrate-Based Controlled Fertigation

During the previous phases I-III, the irrigation and fertilization schemes were preprogrammed and set to run for a duration of 1 to 3 weeks, while the resulted impact on nitrate propagation in the soil was monitored across the soil profile (utilizing sensors 402a-402c). The observations revealed complex relations between the irrigation and fertilization schedule on the nitrate retention and transport within the soil. It became apparent that optimizing nutrient application to improve root uptake while reducing nitrate leaches would require a higher adjusting resolution of both irrigation and fertilization. Consequently, during the fourth phase of the experiment, daily adjustments to both the irrigation and the fertilization schemes were performed based on the measured variations in nitrate concentration and water content across the soil profile.

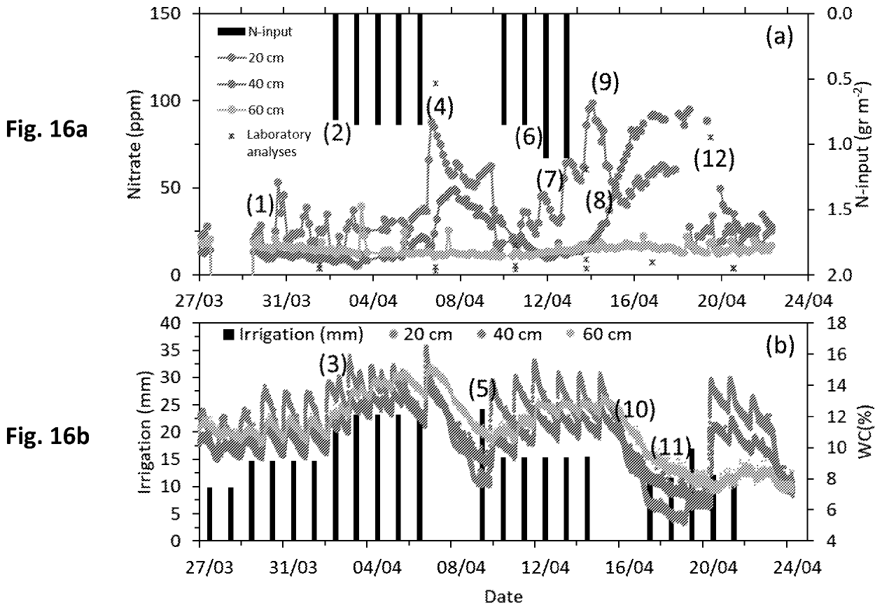

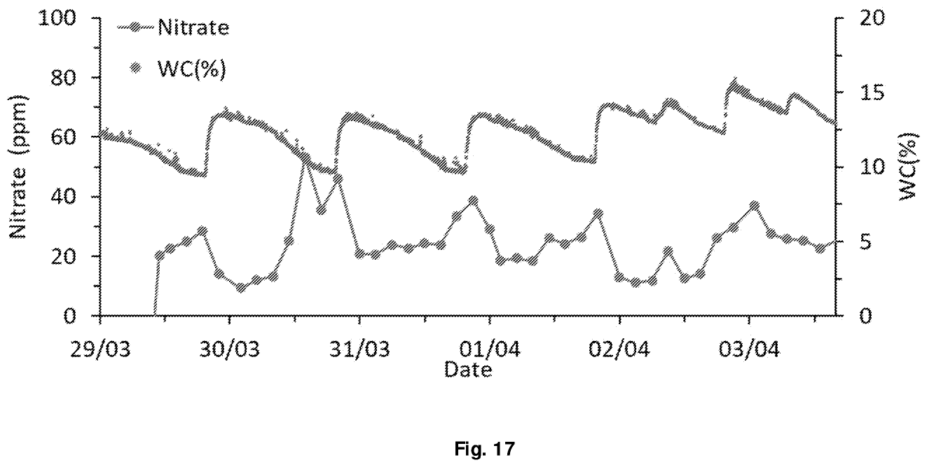

At the early stage of phase IV, following the fertilizer deprivation from the end of phase III, nitrate levels at 40 cm and 60 cm remained relatively low and steady. However, nitrate levels at 20 cm showed daily oscillations (point 1 in FIG. 16a). FIGS. 16a and 16b illustrate phase IV of the controlled irrigation and fertilization experiment: FIG. 16a shows N-inputs to the soil, and continuous nitrate data from 20 cm, 40 cm, and 60 cm; FIG. 16b shows daily irrigation and soil moisture content data at 20 cm, 40 cm, and 60 cm. A closer look at the nitrate fluctuations reveals that they negatively correlate to the soil water content, where each daily irrigation (which conveys a rise in the soil water content) results in a drop in nitrate levels and vice versa (FIG. 17). FIG. 17 shows the nitrate levels and soil water content at 20 cm during phase IV of the controlled irrigation and fertilization experiment. A hydrological explanation for this behavior can be found since porewater found in the soil at the immobile phase can become mobile when the water content levels drop. The introduction of new water to the mobile phase can enrich the monitored solution with nitrate that was previously undetected when found in the immobile phase of the porewater.

To ensure an adequate supply of nutrients to the crop during this stage, a steady supply of 0.85 gr/m² N-fertilizer was applied to the soil, while the irrigation was gradually increased from 10 mm to 23 mm per day (points 2 and 3 in FIGS. 16a and 16b). Following a sharp increase in nitrate levels at 20 cm (point 4 in FIG. 16a), both irrigation and fertigation were stopped to enhance water and nitrate uptake by the plant and prevent down leaching. As a result, nitrate levels dropped at 20 cm, from a peak of about 100 ppm to 70 ppm and later to 20 ppm (point 5 in FIGS. 16a and 16b). Consequently, an immediate rise in nitrate concentration was also observed at 40 cm. As soon as nitrate levels in the depth of 40 cm showed a decrease from 50 ppm to 20 ppm, irrigation and fertilization resumed (points 5 and 6 in FIG. 16). Following this stage, each day was characterized by a peak concentration at 20 cm, which decayed daily until the next fertigation resumed on the next day. The decay in nitrate concentration can result from root uptake, as the crop was at the headling/ripening stage, characterized by high demand for nutrients. This phenomenon can also be explained by moderate down leachate of nitrate, as this event was also followed by a rising concentration of nitrate at 40 cm (point 8 in FIGS. 16a and 16b). Once nitrate levels had reached about 100 ppm at 20 cm (point 9 in FIGS. 16a and 16b), fertilization and irrigation had been brought to a second halt to enhance root uptake and decrease down leaching of leaching nitrate (point 10 in FIGS. 16a and 16b). Yet, to avoid plant stress from water scarcity, moderate irrigation of 8 mm to 10 mm was applied for the remaining 5 days of the experiment (Point 11 in FIGS. 16a and 16b). The irrigation influx indeed reduced the nitrate levels both at 20 cm and 40 cm. However, since the nitrate retention time was sufficient and the plant demands for nutrients were high, no nitrate leaches occurred at 60 cm throughout this current phase of the experiment, indicating zero down leaching of nitrate below the root zone.

The above experiment shows that real-time data relating to the concentration and propagation of nitrate within the soil, together with proper management of fertigation and irrigation, can significantly reduce the excess fertilization that eventually arrives and contaminates the groundwater. Moreover, this object can be achieved without harming the crop. The invention provides a system and method for fulfilling this task without harming the crop. While the experiment was conducted manually, and while manual decisions were made during the experiment, an automatic system can be devised by utilizing: (a) existing protocols that define the minimal amount that the plant actually consumes during various phases of its development and negative feedback designed to minimize the excess of fertilization. The excess of fertilization is measured by (preferably) a real-time system that utilizes one or more sensors to determine nitrate concentration in the soil. The system needs for its operation, namely minimizing excess fertilization, at least one sensor positioned below the plant's roots. However, one or two additional sensors preferable, as they can provide more accurate information about the nitrate concentrations in various soil depths and propagation trends over time. The sensors are typically, but not necessarily, porous interfaces that separately collect water from the soil and transfer a respective water sample to a (preferably) real-time analyzer, providing the respective nitrate concentrations for each sensor.

While some embodiments of the invention have been described by way of illustration, it will be apparent that the invention can be carried into practice with many modifications, variations, and adaptations, and with the use of numerous equivalent or alternative solutions that are within the scope of persons skilled in the art, without departing from the spirit of the invention or exceeding the scope of the claims.

1. A system for reducing down-leaching of nitrate to a region below a crop's roots zone, comprising:
an analysis unit for repeatedly determining a concentration level of nitrate at least at a region below the crop's roots zone, and recording said nitrate concentration levels;
a controller configured to:
(i) receive a recent record of said nitrate concentration level below the roots zone and at least one previous record of concentration level, and determine a rate of change between said recent and previous records; and
(ii) based on said rate of nitrate concentration change, activating fertigation and irrigation in times and periods that minimize the down-leaching of nitrate to below the roots zone;
wherein said system comprises at least one water-sample collecting sensor positioned below the crop's roots zone that transfers the sample to said analysis unit.

2. A system according to claim 1, wherein for a grain crop, vegetable crops, and greenhouse crops, said below the roots water-sample collecting sensor is positioned at a depth of between 50 cm to 70 cm below the ground surface, and for trees, said below the roots sensor is positioned at a depth of between 80 cm to 100 cm below the ground surface level.

3. A system according to claim 1, further comprising at least one additional water sample-collecting sensor positioned at the crop's roots zone, said sensor further transfers water samples to said analysis unit for further determination of nitrate concentration at the roots zone, and wherein said controller further considerers the level of nitrate concentration at the roots zone in its times and periods management of fertigation and irrigation configured to minimize the flow of nitrate to below the roots zone.

4. A system according to claim 3, wherein for grain crops, vegetable crops, and greenhouse crops, said water-sample collecting sensor within the roots zone is positioned at a depth 30 cm to 50 cm below the ground surface, and for trees, said sensor within the roots zone is positioned at a depth of between 40 cm to 60 cm below the ground surface.

5. A system according to claim 3, further comprising at least one additional water sample-collecting sensor positioned above the crop's roots zone, said sensor further transfers water samples to said analysis unit for further determination of nitrate concentration above the roots zone, and wherein said controller further considerers the level of nitrate concentration above the roots zone in its times and periods management of fertigation and irrigation configured to minimize the down-leaching of nitrate to below the roots zone.

6. A system according to claim 5, wherein for grain crops, vegetable crops, and greenhouse crops, said water-sample collecting sensor above the roots zone is positioned at a depth 10 cm to 30 cm below the ground surface, and for trees, said sensor above the roots zone is positioned at a depth of between 20 cm to 40 cm below the ground level.

7. A system according to claim 1, comprising a set of three water-sample collecting sensors, wherein for grain, vegetable crops, and greenhouse crops, the sensors are positioned at depths of 10 cm-30 cm, 30 cm to 50 cm, and 60 cm and 50 cm to 70 cm, and for fruit trees the sensors are positioned at depths of 20-40 cm, 40-60 cm, and 80 cm to 100 cm, respectively.

8. A system according to claim 1, further comprising one or more soil moisture sensors, each sensor provides soil water content data at each depth, respectively, for consideration in the management of the fertigation and irrigation that minimizes the down-leaching of nitrate to below the roots zone.

9. A system according to claim 7, wherein a plurality of sets of water sample collecting sensors are positioned along a crop field and wherein the system averages concentration results of a plurality of sensors, respectively, that are positioned at the same depths.

10. A system according to claim 1, wherein each said water-sample collecting sensor comprising a porous interface and wherein the water sample is transferred to an optical flow cell within the analysis unit utilizing a tube.

11. A system according to claim 1, wherein said analysis unit operates in real-time.

12. A system according to claim 11, comprising closed-loop fertigation and irrigation management.

13. A system according to claim 1, wherein said analysis unit operates off-line.

14. A system according to claim 1 wherein:
during a training stage the system is operated in a closed loop to build a model defining a dependency of down-leaching of nitrate to below the roots zone on periods and amounts of irrigations and fertilizations, respectively; and
during an operational stage, a control unit operates in an open-loop without sensors and analysis unit, applying irrigations and fertilizations following a plan prepared based on said model, said plan is configured to minimize down-leaching of nitrate to a region below a crop's roots zone.

15. A system according to claim 1, further utilizing weather and rain data to optimize the management of fertigation and irrigation.

16. A method for reducing down-leaching of nitrate to a region below a crop's roots zone, comprising:
positioning at least one water-sample collecting sensor below the crops' roots zone;
optionally positioning at least one additional water-sample collecting sensor at or above the crops roots zone;
receiving water samples from said water-sample collecting sensors, and repeatedly determining a concentration level of nitrate at least at a region below the crop's roots zone, and possibly also at the roots zone and above the roots zone, and recording said concentration levels;
based on said determination of nitrate concentration at least below the roots zone, and previous one or more recordings of nitrate concentration below the roots zone, determining a rate of concentration change below the roots zone; and
based on said rate of change, managing fertigation and irrigation in times and periods that minimize nitrate down-leaching to the region below the roots zone.

17. The method of claim 16, further positioning one or more wetness sensors, at locations selected from below the roots zone, at the roots zone, and/or above the roots zone, and considering wetness data acquired by these sensors for said irrigation and fertigation management.

18. The method of claim 16, further considering weather data for said irrigation and fertigation management.

19. The method of claim 16, wherein said management comprising:
determining a nitrate concentration below the roots zone, and optionally also at or above the roots zone, and recording said determinations;
comparing between a current nitrate concentration below the roots zone and a previous nitrate concentration determination below the roots zone and determining a rate of change in the nitrate concentration;
comparing said rate of change to a predefined threshold scale;
if said rate of change is found to be high, performing one or more of postponing, reducing, or skipping the next irrigation and/or fertigation; or
if said rate of change is found to be low or zero, continue the irrigation and fertigation according to a regular protocol.

20. The method of claim 19, further considering nitrate concentration determinations at or above the roots zone for said irrigation and fertigation management.

21. A method according to claim 16 further comprising:
during a training stage, applying the method in a closed loop and building a model defining a dependency of down-leaching of nitrate to below the roots zone on periods and amounts of irrigations and fertilizations, respectively; and
during an operational stage, operating in an open-loop without positioning said one or more sensors, and without determining said nitrate concentrations and rate of change, while applying irrigations and fertilizations following a plan prepared based on said model, said plan is configured to minimize down-leaching of nitrate to a region below a crop's roots zone.
